(19) United States
(12) Patent Application Publication
(10) Pub. No.: US 2007/0175005 A1
(43) Pub. Date: Aug. 2, 2007

(54) Adjustable buckle with dual tensioning and releasable closure for joining opposite ends of an article

(75) Inventor: Miguel J. Latronica, Chicago, IL (US)

Correspondence Address: PERRY HOFFMAN & ASSOCIATES P.C., PO BOX 1649, DEERFIELD, IL, 60015, US

(73) Assignee: WHO WOULD OF THUNK IT INNOVATIONS, LLC, Chicago, IL (US)

(21) Appl. No.: 11/715,640

(22) Filed: Mar. 8, 2007

Related U.S. Application Data
(63) Continuation-in-part of application No. 11/485,121, filed on Jul. 12, 2006.
(60) Provisional application No. 60/698,753, filed on Jul. 13, 2005.

(51) Int. Cl. A44B 11/00; A61F 5/00

(52) U.S. Cl. 24/164; 24/198; 602/20

(57) ABSTRACT

An advantageous dual tensioning buckle facilitates application in sporting apparel, safety, backpacking, child safety, and various other article strapping uses and the like for variably adjusting the tensioning of articles from either end of the buckle while also providing a releasable closure. The buckle includes a socket and a plug capable of releasably joining together and having a low profile for convenient use in numerous applications. The socket and plug are capable of receiving a first and second length of material respectively; and a non-fabric sewable static strap is integrally molded or affixed to each of the socket and plug and sewable onto a garment with the first and second lengths of material extending from within the garment for adjustable tensioning while also providing a releasable closure.

### CROSS-REFERENCE TO RELATED APPLICATION

This application is a Continuation in Part of U.S. patent application Ser. No. 11/485,121 filed on Jul. 12, 2006, which application claims priority pursuant to 35 U.S.C. 119 (e) to U.S. Provisional Application No. 60/698,753 filed on Jul. 13, 2005 which are incorporated herein by reference in their entirety.

### BACKGROUND OF THE INVENTION

1. Field of the Invention

The invention relates to closure devices, and more particularly to a buckle which variably adjusts the tensioning of an article from either end of the buckle while also providing a releasable closure. The invention also relates to a method for improving a garment article through the adjustable dual tensioning and releasable closure improvements of the buckle of the invention.

2. Background of the Invention

Poor posture is a serious problem afflicting millions of Americans and contributes to numerous health problems such as back aches, hunched backs, rounded shoulders, tension headaches, and overall poor health and inflexibility of the spine. Poor posture is so wide spread that solutions to this problem have been attempted by many different sources, including medical companies selling therapeutic support devices and fitness companies selling posture training devices.

The therapeutic support devices sold in the marketplace attempt to relieve some of the painful effects of poor posture, such as an aching back for example, by supporting and somewhat immobilizing specific parts of the back, neck or shoulders, in an effort to relieve some of the user's discomfort. These therapeutic support devices are not effective in training the user to achieve and maintain proper posture because the devices simply restrict a user from moving specific body parts in order to relieve discomfort, and do not retrain muscles, or the user, to maintain proper posture. Further, excessive use of an upper and/or lower body muscle support device could result in the eventual weakening of some upper body muscles from their non-use while wearing the device, potentially worsening the user's posture.

The known posture training devices focus mainly on helping the user achieve a proper position to improve the user's performance in a specific activity, such as golf for example. These devices are not practical or effective in training the user to achieve and maintain proper posture while working or resting, because they are cumbersome or impractical to wear, sometimes too heavy to wear for any length of time, not easy to adjust or they simply are not intended to be worn beyond participation in the specific activity or training session. Also, many of the known devices contain complicated signal and feedback transmitters and circuitry to provide feedback relating to the user's posture to the user, or a third party, rather than training the user to recognize the feel of their own body as it achieves and maintains proper posture, and create muscle memory in the user when proper posture is achieved.

Additionally, many of the above described devices as well as other articles and garments are either non-adjustable, or difficult and cumbersome to adjust while worn on the user. Many garments and other articles are either adjusted by standard buckles or velcro, or they are not adjustable at all, and instead provide some elastic sections or parts which stretch and enable a user to wiggle into the garment or device.

The known buckles are also limited by either their bulkiness or lack or versatility, in that do not allow adjustment of the tensioning of the article or they only allow an article or garment to adjust from one side, and none of the know buckles are versatile enough to variably adjusts the tensioning of an article, garment, or length of material from either end of the buckle while also providing a releasable closure.

### SUMMARY OF THE INVENTION

The invention addresses shortcomings of the prior art to provide a buckle which adjusts the tensioning of an article from either end of the buckle and provides a releasable closure. The buckle of the invention includes a first and second static strap affixed to either end of the releasable buckle, respectively, thereby allowing the buckle to receive first and second lengths of material, respectively, providing for quick and easy adjusting of the tensioning of the material from either end of the buckle extending from within the article. Additionally, the article may be received into an outer garment for adjustable tensioning and releasable closure thereof.

In one embodiment of the invention, the buckle includes a socket affixed to a first static strap and a plug affixed to a second static strap and the socket and plug are each capable of receiving a first and second length of material, respectively. The socket and plug are releasably engaging and the first and second straps are each sewable onto a garment with the first and second lengths of material extending from within the garment for adjustable tensioning by either the first or second length of material.

In another embodiment of the invention, the socket includes an inlet and a resilient latch member projecting into the inlet, and the plug includes a resilient engaging member capable of releasably coupling with the latch member of the socket. The socket and plug are each capable of being sewn onto the garment to provide a releasable closure.

In another embodiment, the socket is a low profile hollow socket having a front shell with a resilient pressuring member formed in the front shell operable with the resilient latch member and capable of engaging with the plug which is also low profile.

In yet another embodiment, the first and second static straps couple to opposite ends of a band for adjustable tensioning of the band from either end with the socket and plug each capable of being sewn onto the garment to provide a releasable closure.

In another embodiment of the invention, the article may be received into an outer garment for adjustable tensioning and releasable closure thereof.

In another embodiment, a method for improving a garment article with adjustable tensioning and releasable closure includes providing a socket and a plug each capable of receiving a first length of material and a second length of material respectively, the first and second static straps each being sewable onto the garment with the first and second length of material extending from within the garment for adjustable tensioning. The socket and plug are each capable of being sewn onto the garment to provide a releasable closure.

### BRIEF DESCRIPTION OF THE DRAWINGS

For the purpose of facilitating an understanding of the inventions, the accompanying drawings and description illustrate a preferred embodiment thereof, from which the inventions, structure, construction and operation, and many related advantages may be readily understood and appreciated.

FIG. 1 is a perspective view of a posture band of the invention illustrating a body including a partial second layer and a positioning mechanism having a first and second layer;

FIG. 2 is a perspective view of an embodiment of the posture band in use illustrating a buckle in open and closed positions;

FIG. 3 is another perspective view of the posture band embodiment of FIG. 2 illustrating the positioning mechanism in use and the semi-rigid insert;

FIG. 4 is a perspective view of an embodiment of the posture band in use illustrating a loop of the elastomeric body protruding from a covering for easy adjustment of the posture band while in use;

FIG. 5 is a perspective view of the buckle illustrating a first side released from a second side;

FIG. 6 is a perspective view of an embodiment of the posture band in use illustrating a foam insert or the like coupled to the positioning mechanism;

FIG. 7 is a perspective view of the posture band embodiment illustrating use with a portable music player;

FIG. 8 is a perspective view of the posture band embodiment illustrating use with a flap cover structure covering the buckle, ends of the posture band body and for securing personal accessories and effects;

FIG. 9A is a perspective view of a socket of the invention;

FIG. 9B is a back view of the socket of the invention;

FIG. 10 is a cross sectional view of the socket along line A of FIG. 9B illustrating the resilient latch member;

FIG. 11A illustrates a non-fabric sewable static strap integral with the socket;

FIG. 11B illustrates the non-fabric sewable static strap staked to the socket;

FIG. 11C illustrates the non-fabric sewable static strap affixed to the socket opposite a bar or post;

FIG. 12 is a perspective view of a plug of the invention;

FIG. 13 is a side view of the plug of the invention illustrating the resilient engaging member;

FIG. 14A illustrates the non-fabric sewable static strap staked to the plug;

FIG. 14B illustrates the non-fabric sewable static strap affixed to the plug opposite a bar or post;

FIG. 15 is a perspective view of the non-fabric sewable static strap of the invention;

FIG. 16 is a back plan view of the non-fabric sewable static strap;

FIG. 17 is a side view of the non-fabric sewable static strap;

FIG. 18 illustrates the band sandwiched between two or more layers of fabric; and

FIG. 19 illustrates guide loops keeping the band in place.

### DETAILED DESCRIPTION OF THE PREFERRED EMBODIMENTS

The following description is provided to enable those skilled in the art to make and use the described embodiments set forth in the best modes contemplated for carrying out the invention. Various modifications, however, will remain readily apparent to those skilled in the art. Any and all such modifications, equivalents, and alternatives are intended to fall within the spirit and scope of the invention.

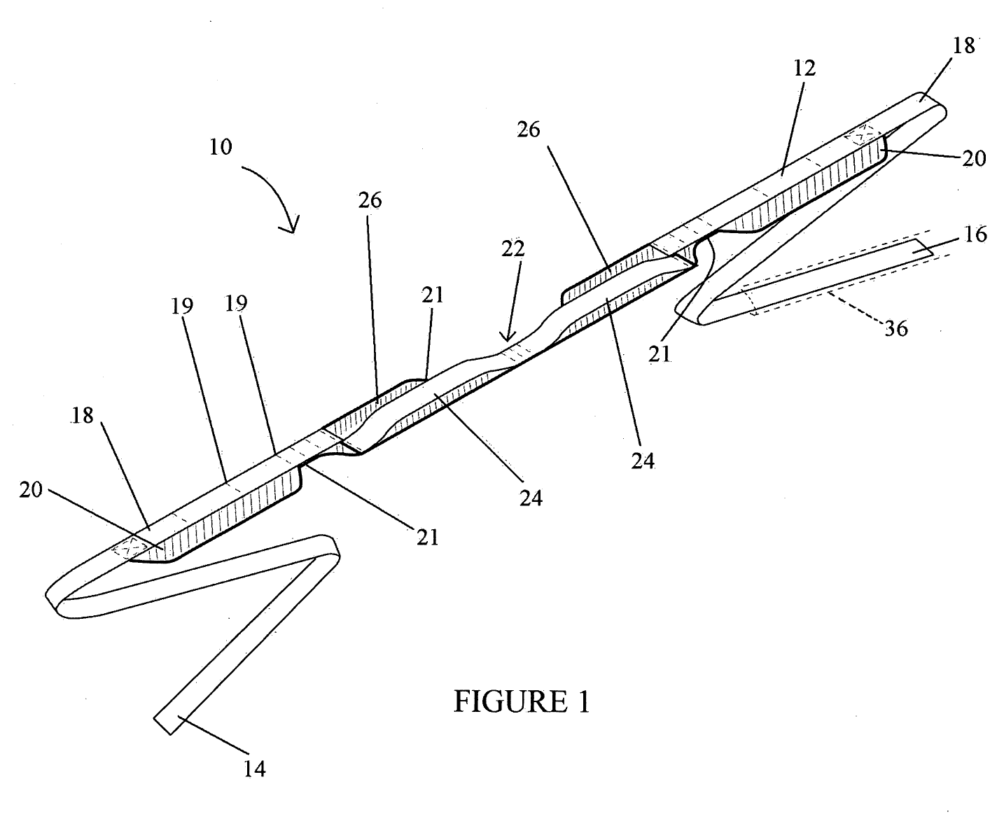

A posture band 10, as seen in FIG. 1, includes a body 12 having a first end 14 and second end 16. The body 12, as seen in FIG. 1, has a general length and a width and can be made to a variety of length and width dimensions to comfortably fit user's of any size and shape, including large adults and children. Alternatively, it is also contemplated that the body 12 is circular in shape and therefore not have any ends at all, as seen in FIG. 4.

The body 12, as seen in FIG. 1, includes a first layer 18, and a second layer 20. One of the first and second layers, 18 and 20, or alternatively, both layers are manufactured from any variety of elastomeric materials including elastic and rubber, etc., and each of the first and second layers, 18 and 20 may include the same or different elastomeric materials, in any combination thereof. It is also contemplated that the body 12 can include only one layer of elastomeric material or, alternatively, more than two layers of elastomeric or non-elastomeric material, in combination.

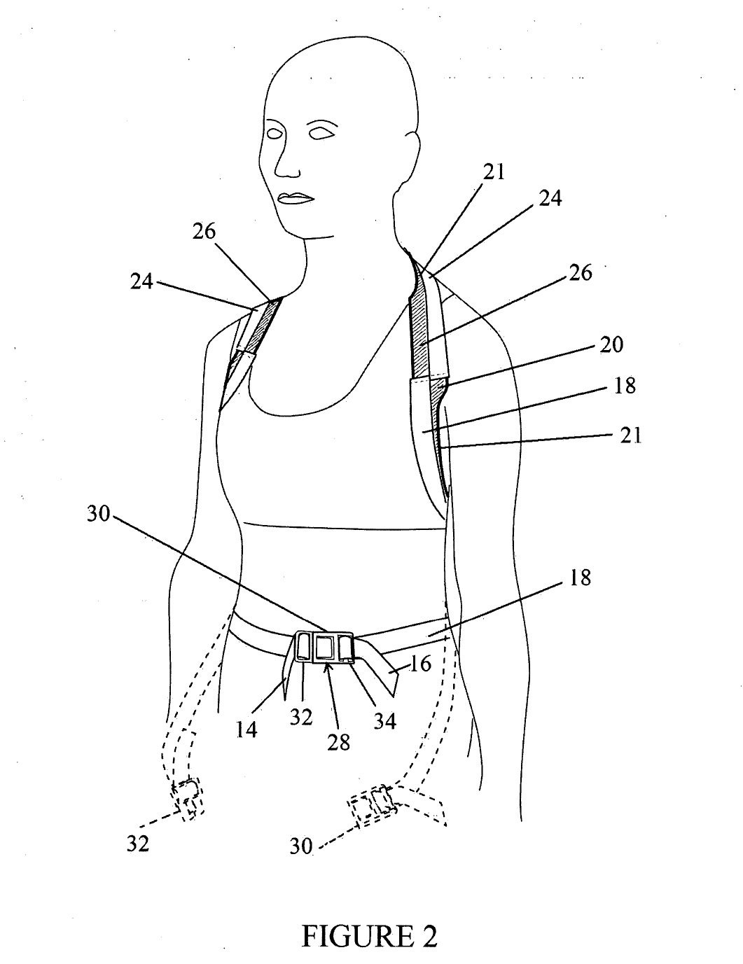

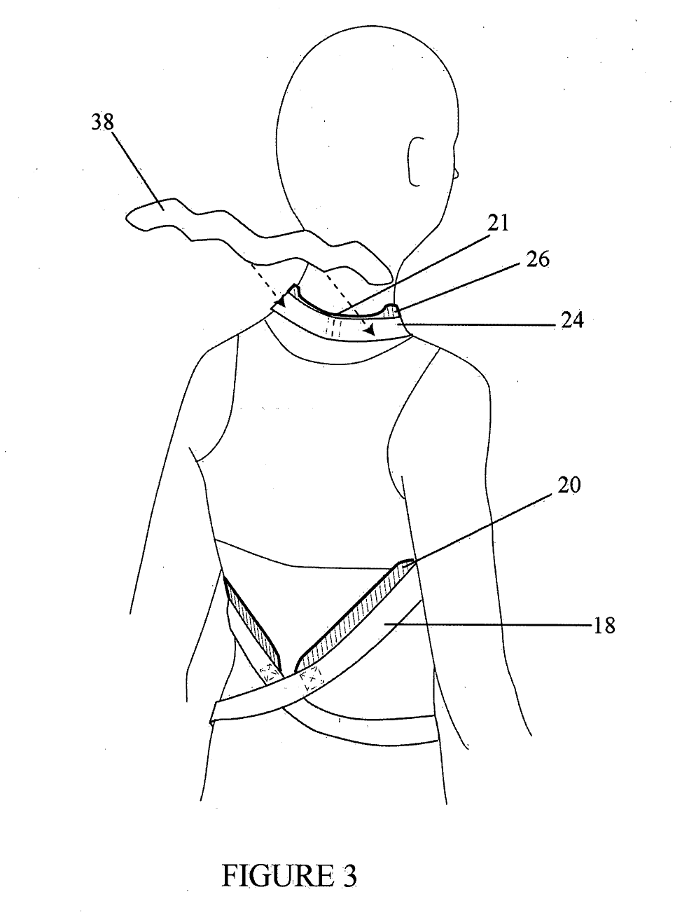

A positioning mechanism 22, seen in FIG. 1, is coupled to the body 12 for facilitating the proper alignment and tensioning of the body on a user, as seen in FIGS. 2 and 3. The positioning mechanism also includes a first layer 24 and a second layer 26, as seen in FIG. 1. One of the first and second layers, 24 and 26, or alternatively, both layers, 24 and 26 are manufactured from any variety of elastomeric materials including elastic and rubber, etc. and each of the first and second layers, 24 and 26 may include the same or different elastomeric materials, in any combination thereof. Similar to the body 12, it is also contemplated that the positioning mechanism 22 can include only one layer of elastomeric material or, alternatively, more than two layers of elastomeric or non-elastomeric material, in combination.

As is also seen in FIG. 1, second layer 26 of the positioning mechanism 22 is integral with second layer 20 of the body 12, and first layer 24 of the positioning mechanism 22 and first layer 18 of the body 12 are sewn together. Layers 24 and 26 of the positioning mechanism 22 can be secured to layers 18 and 20 of the body 12, respectively, by any means known in the art, for example, sewing, riveting, and gluing, etc., and each may alternatively be integral with the body 12, or any combination thereof.

In use, the body 12 wraps around the user, as seen in FIGS. 2-4, and further around the stomach of the user, as seen in FIGS. 2 and 3. The positioning mechanism 22 rests on the back of the neck of the user, as seen in FIGS. 2-4, training the user to externally rotate his/her shoulders back bringing the shoulder blades inferior and medial toward a longitudinal midline of the user and holding up his/her head. Wearing of the posture band 10 of the invention will train the user's muscles to achieve and maintain proper posture, as described above, and create a memory in the user's muscles to maintain this proper posture even when the user is not wearing the posture band.

The Body 12 further incorporates one or more recesses 21 cut into the width of the second layer 20, as seen in FIG. 1, to facilitate the proper positioning of the body on the user as seen in FIGS. 2 and 3. As seen in FIG. 1, two recesses 21 are cut into the width of layer 20, one on either side of the coupled positioning mechanism 22.

Further, the positioning mechanism 22 includes a recess 21 cut into the width of second layer 26, as seen in FIG. 1, also to facilitate the proper positioning of the positioning mechanism 22 on the back of neck of the user, as seen in FIGS. 2 and 3. It is also contemplated that the recesses 21 can extend into first layers 18 and 24, respectively, or alternatively, a recess may be cut into first layers 18 and 24, instead of second layers 20 and 26, respectively, at points which would facilitate the positioning of the body 12 and positioning mechanism 22 on the user.

Second layer 20 of body 12 and second layer 26 of positioning mechanism 22 also have a wider width than first layers 18 and 24, respectively, as seen in FIG. 1. The wider width of second layers 20 and 26 facilitates the varied tensioning and the correct positioning and fit of the band on the user, as seen in FIGS. 2 and 3, and second layer 20 also provides support for the lower back of the user while in use, as seen in FIG. 3. The reverse width configuration is alternatively contemplated, with first layers 18 and 24 being wider than second layers 20 and 26, respectively.

The first layer 24 of positioning mechanism 22 also has a longer length than second layer 26, as seen in FIG. 1, providing a variable tension along the positioning mechanism as it is stretched while in use. The first layer 24 also includes an elastomeric material which is more difficult to stretch as compared to second layer 26, also providing a variable tension along the positioning mechanism as it is stretched while in use. Additionally, a variety of stitching lines 19 may also be included along the length of the body 12, as seen in FIG. 1, to provide a specific amount of tension directed along specific sections of the body 12 as it is stretched while in use.

As seen in FIG. 2, a buckle 28 may be coupled to both first end 14 and second end 16 of the band. The buckle 28 includes a socket 32 and a plug 34 which can alternatively be described as a first side 32 and a second side 34. A hooking mechanism 30 (described in more detail below) releasably couples the plug 34 to the socket 32 and can be simply opened and closed, as shown in FIG. 2, to easily release-the tension of the band and/or remove it from the user.

The unique design of the buckle 28 of the invention allows both ends 14 and 16 to provide means for adjusting the tensioning of the body 12 while in use. Either end 14 or 16 can be pulled tighter or loosened up to facilitate the correct fit of the body 12 on the user while the user is wearing the band. This unique design allows a user to continuously wear the posture band of the invention as a user transitions from resting to playing and/or working because the band can to be quickly and simply loosened or tightened while worn by the user, thus adjusting the specific amount of tension exhibited by the band when worn during various activities.

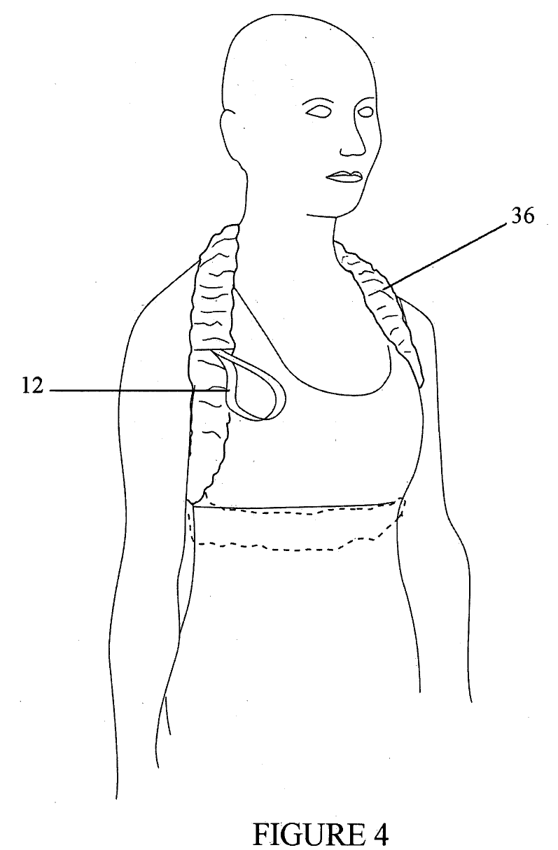

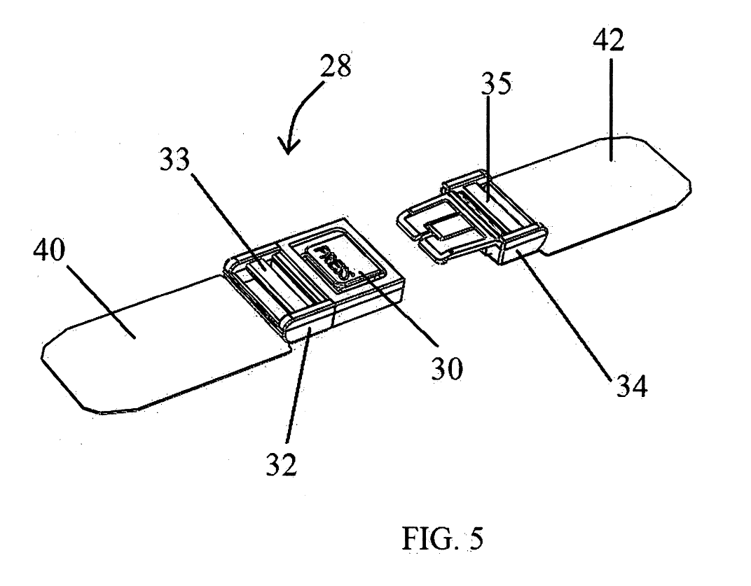

As seen in FIGS. 1 and 4, a covering 36 incorporates a substantial portion of the body 12 therein. As seen in FIG. 5, a first non-fabric sewable static strap, or alternatively, a stiffening plate 40 is attached to the first end 32 and a second non-fabric sewable static strap, or alternatively, a stiffening plate 42 is attached to second end 34 and coupled to the covering 36 by sewing, gluing, riveting, etc, or other means known in the art. Alternatively, the static straps 40 and 42 be may integral with ends 32 and 34 respectively. Omitting ends 14 and 16 of the body 12, as well as the buckle 28, from incorporation into the covering 36 however, will further facilitate the ease with which a user can adjust the band while in use, as described above.

The covering 36 is manufactured from a wearable material such as silk, cotton, polypropylene, etc., or any combination thereof. The polypropylene material is particularly useful if the posture band 10 is to be worn during exercising. The covering 36 provides a more comfortable feel for the user and is also fashionable and can include a stripe or poka-dot pattern, allowing the user to wear the band on top of as well as underneath their clothing.

The covering 36 may also contain openings for providing access to the inside surrounding the body 12 contained therein, whereby cords for electronic devices such portable digital music players, DMPs, DVDs, IPods™, MP3 players, and the like, worn on the user, can be threaded through and contained largely within the cover 36, as seen in FIG. 7. Thus the covering 36 may further facilitate use of the body 12 as being capable of incorporating use with such portable music players. The ability to contain and conceal cords of the type described above enhances the users comfort when wearing such devices along with the posture band 10.

A flap 48 may also be coupled to the posture band 10, either removably or non-removably, by any means known in the art, including Velcro, sewing, gluing, riveting etc., to conceal the buckle 28 and ends 14 and 16 for a more aesthetic look when in use. The flap 48 structure is generally a rectangular shape and secures around the body 12 as described for use as a cover or holding structure covering the buckle, ends of the posture band body and for securing personal accessories and effects, etc. The flap 48 can be any variety of shapes and sizes and made of any variety of materials such as polypropylene or similar material having enough structure to hold identification cards and/or a money compartment etc. and yet flexible enough to be comfortably worn by a user, as seen in FIG. 8. The flap 48 is also an excellent location for personal identification markings, or advertising, or other personal effects.

A loop of the body 12 protrudes from the covering, as seen in FIG. 4, provides another means for adjusting the tensioning of the body 12 while in use. As seen in FIG. 4, the body 12 can be circular in shape and tightening or loosening the band while in use can be accomplished by pulling on or loosening up the exposed body 12. Additionally, a means for securing the desired tensioning of the band, once achieved, may include a button and slot arrangement located on the exposed loop of the body 12 to adjust and secure the correct size and fit of the band on the user.

The posture band 10 further includes a first semi-rigid insert 38 which is positioned adjacent the body 12 to enhance the effect of the positioning mechanism 22, as seen in FIG. 3. The insert 38 can be any variety of shapes and sizes, and has a generally longer length then its width, similar to the positioning mechanism 22, as seen in FIG. 3. The insert 38 is manufactured from any variety of semi-rigid material such as plastic or resin, etc., or any combination thereof.

As seen in FIG. 3, the insert 38 is located adjacent the positioning mechanism 22 and may be coupled either removably or non-removably, to the positioning mechanism 22 by any means known in the art. The first insert 38 can be at least partially contained within or may be provided as a profile or template for the covering 36 providing an alternative means for holding the insert 38 in place and providing a comfortable feel for the user. Further, a second semi-rigid insert may be coupled either removably or non-removably to the first semi-rigid insert 38 by riveting, etc. or any other securing means known in the art. The second semi-rigid insert may be manufactured from the same materials and may be generally the same shape and size as insert 38, or alternatively, a somewhat smaller size but generally the same shape as insert 38 further enhancing the effect of the positioning mechanism 22 while in use.

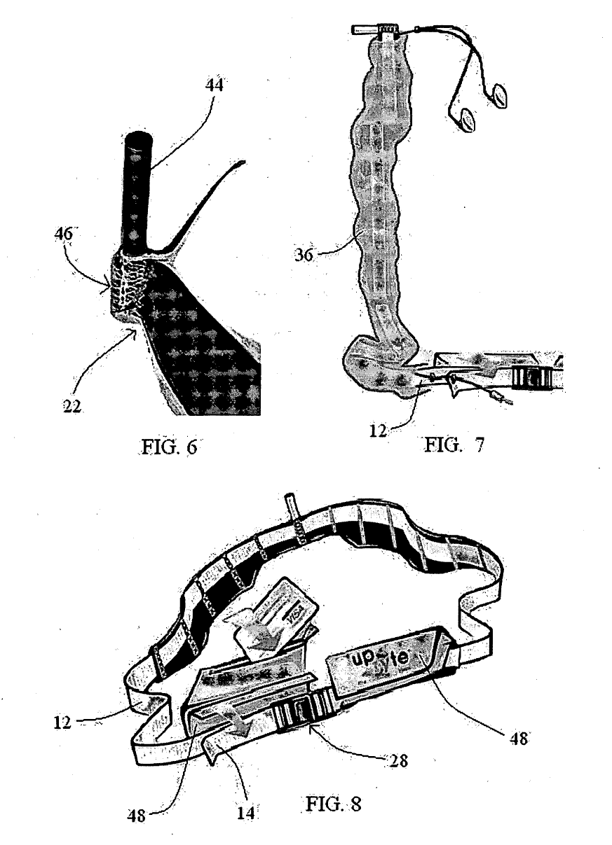

The posture band 10 further includes a foam insert 44 located adjacent the positioning mechanism 22 and may be removably coupled to the positioning mechanism 22 by any means known in the art, such as Velcro sewing, gluing, riveting; etc. As seen in FIG. 6, the foam insert 44 is generally small, about 1½ inches tall and is removably inserted into a loop portion 46 of the positioning mechanism 22. It is also contemplated that the foam or like material can be any variety of shapes and sizes and can be permanently attached to the body 12 or positioning mechanism 22. When in use, the foam portion 44 provides the user with a gentle tactile reminder to the lift up his/her head.

A method for improving a user's posture includes the steps of providing a posture band having a body made of an elastomeric material, coupling a positioning mechanism to the body and wrapping the body around the back of the neck, over both shoulders and around the back of the user. Adjusting the positioning mechanism of the band such that the positioning mechanism rests on the back of the neck of the user trains the user to roll his/her shoulders back bringing the shoulder blades together and holding up his/her head. Additionally, cutting one or more recesses into a width of the body facilitates the proper positioning of the body on the user.

Providing a first layer and a second layer of elastomeric material coupled together as the positioning mechanism facilitates the proper tensioning of the body on the user, and lengthening only one of the first or second layers of the positioning mechanism provides for a varying tension along the positioning mechanism as it is stretched while in use. Further coupling a semi-rigid insert to the body enhances the effect of the postioning mechanism

Providing a buckle coupled to both a first end and a second end of the body provides a means for adjusting the tension of the body while in use from either of the first or second ends. Tightening or loosening either end of the body facilitates the correct fit on the user allowing the user to continuously wear the posture band of the invention while transitioning from resting to playing and/or working thus simply and efficiently adjusting the specific amount of tension exhibited by the band when worn during various activities.

Incorporating a substantial portion of the body within a covering provides a more comfortable and fashionable feel for the user, however omitting the buckle and first and second ends of the body from the covering will still allow for the easy and efficient adjusting of the tensioning of body while worn by the user, as described above.

The good health and flexibility of a person's spine can greatly enhance that persons′ overall health. Likewise a person's posture and alignment of their spine significantly influences a person's long term health and well being. When the posture band 10 of the invention is worn it is believed to stimulate the proprioceptive function of the mechanoreceptors of the muscles, so that the tension of the band (which is pulling in the direction of the less active postural muscles (primarily but not limited to the middle and lower trapezius and serratus anterior muscles) increases the activity of the less active muscles, and assists the overused/over active muscles (primarily but not limited to the pectoralis major, pectoralis minor, upper trapezius and levator scapulae muscles) to relax by having the tension of the band pulling in the opposite direction of the contraction vector of the overactive/overused muscles and likely also stimulating a reciprocal inhibition effect.

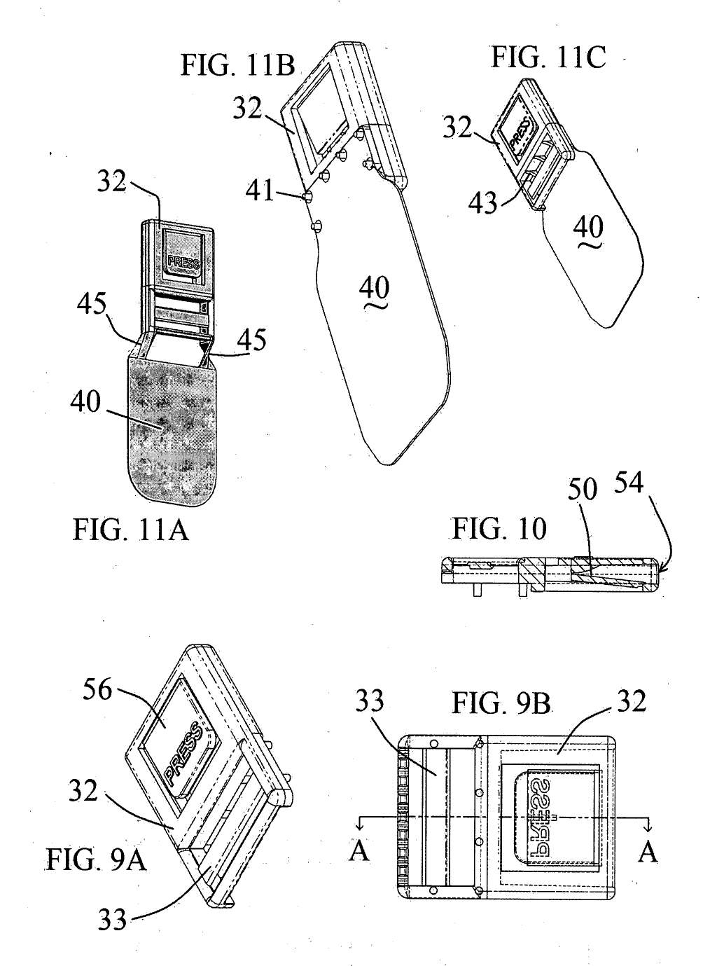

The buckle 28, as seen in FIGS. 9 through 19, can releasably join opposite ends of any article, garment, length of material, or outer wear, including the band 10 as discussed above. The buckle 28 provides advantageous application in sporting apparel, safety, backpacking, child safety, and various other article strapping uses and the like. The buckle 28 includes the socket 32 and plug 34 which are capable of releasably joining together, as discussed above. The hooking mechanism 30, as seen in FIGS. 5 and 9-13, includes a resilient latch member 50 coupled to the socket 32 and operable with a resilient engaging member 52 coupled to the plug 34. Socket 32, as seen in FIGS. 9A-10, is hollow and has an inlet 54 in which the resilient latch member 50 extends. When the plug 34 is inserted into the inlet 54, the resilient engaging member 52 of the plug releasably engages with the resilient latch member 50 of the socket.

The socket 32, as seen in FIG. 9A, includes a back shell, a top shell, a bottom shell, and two side shells, with a resilient pressuring button member 56 formed in the front shell and the resilient latch member 50 projecting therein. Depressing the pressuring button member 56 releases the engaging member 52 from the latch member 50 when uncoupling of the buckle 28 is desired.

The socket 32 includes a bar or post 33 coupled to or integral with the socket at an end opposite the inlet 54, as seen in FIG. 9A, and is capable of receiving a first length of material. A first and second opening lie on either side of the bar or post 33 allowing the first length of material to be threaded through the first opening around the bar or post and back through the second opening to adjustably fasten the first length of material to the socket.

Likewise, the plug 34 includes a bar or post 35 coupled to or integral with the plug at an end opposite the resilient engaging member 52, as seen in FIG. 12, and is capable of receiving a second length of material. A first and second opening lie on either side of the bar or post 35 allowing the second length of material to be threaded through the first opening around the bar or post and back through the second opening to adjustably fasten the second length of material to the plug.

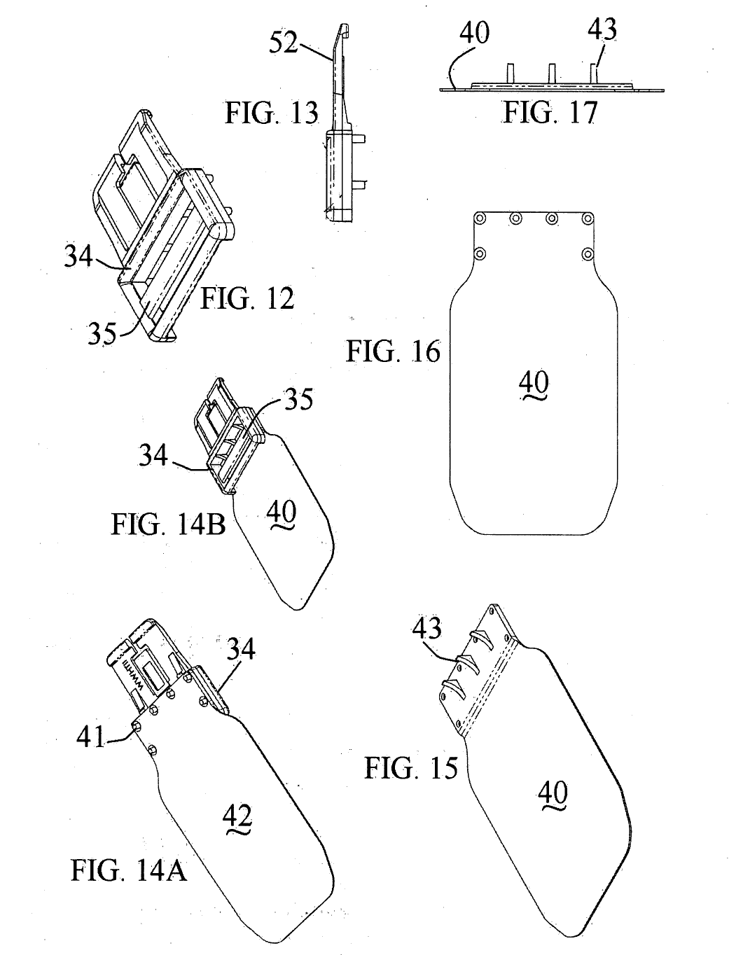

First non-fabric sewable static strap 40 is affixed to socket 32 at an end opposite the inlet and adjacent the bar or post 33, as seen in FIGS. 11A-C, and second non-fabric sewable static strap 42 is affixed to plug 34 at an end opposite the engaging member and adjacent the bar or post 35, as seen in FIG. 14A-B. Static straps 40 and 42 are positioned such that they do not interfere with the hooking mechanism 30 allowing for easy coupling and uncoupling of the socket 32 and plug 34, as well as bars 33 and 35 allowing for easy tensioning of the first and second lengths of material.

In the presently described embodiment, as seen in FIG. 11A, static strap 40 is integrally molded to socket 32, by means such as injection molding or other similar means, etc. Similarly, static strap 42 is integrally molded to plug 34 in the presently described embodiment. In an alternative embodiment, as seen in FIGS. 11B-C and 14B-C, static straps 40 and 42 are affixed to socket 32 and plug 34 respectively, by means such as heat staking with stakes 41 clearly shown in FIGS. 11B and 14A. Further, in other alternative embodiments static straps 40 and 42 are affixed to socket 32 and plug 34 respectively, by means such as gluing, riveting, etc.

Static straps, 40 and 42 are generally flat having a thin profile, as seen in FIGS. 11A and 15-17, facilitating the ease with which an article or garment is sewn to the straps. As seen in FIGS. 11A and 15-16, the straps of the invention have a generally rectangular shape but can alternatively take any shape sized and configured to allow an article or garment to be firmly affixed to the straps.

In the presently described embodiment, as seen in FIG. 11A, static strap 40 is slightly apart from socket 32 allowing for easy and efficient. threading of the first length of material through the first opening around bar or post 33 and back through the second opening to adjustably fasten the first length of material to the socket 32. In the presently described embodiment, static strap 40, as seen in FIG. 11A, is integrally molded to extension members 45 which are integrally molded to socket 32, such that members 45 position strap 40 slightly apart from socket 32. Members 45 may also be slightly offset from socket 32, as seen in FIG. 11A, causing strap 40 to be offset and on a different planer surface than socket 32.

In an alternative embodiment, as seen in FIGS. 15 and 17, ridges 43 are affixed to straps 40 and 42 and positioned at the ends of straps 40 and 42 which are affixed to socket 32 and plug 34 respectively. As seen in FIG. 11C, ridges 43 of strap 40 extend into the opening adjacent bar 33 and help facilitate the threading of the first length of material onto the socket 32. Similarly, ridges 43 of strap 42 extend into the opening adjacent bar 35 and help facilitate the threading of the second length of material onto the plug 34.

In use, first and second static straps 40 and 42 respectively, are each sewable onto a garment with the first and second lengths of material extending from within the garment for adjustable tensioning. The first length of material is threaded through the socket 32 and the second length of material is threaded through the plug 34, as described above, providing for the tensioning of the length of material by either the first length or the second length or both. The socket 32 and plug 34 are each capable of being sewn onto the garment to provide a releasable closure. Additionally, an outer garment such as a suit or sports jacket, vest, apron, or any other upper torso clothing or body wear can receive the article or garment therein for adjustable tensioning and releasable closure thereof.

First and second static straps 40 and 42 respectively, can also be coupled to opposite ends of the band 10, as discussed above for adjustable tensioning of the band 10 from either end with the socket 32 and plug 34 each capable of being sewn onto the garment to provide a releasable closure. Both the socket 32 and plug 34 comprise a low profile providing a non-bulky, convenient and sleek buckle capable of incorporating into any garment or outer garment. The buckle 28 can also be retractable, similar to a retractable cord lock inside of a jacket or backpack, such that when the band 10 is engaged the buckle 28 is out of sight but easily retrievable with a center release for easy tensioning and release.

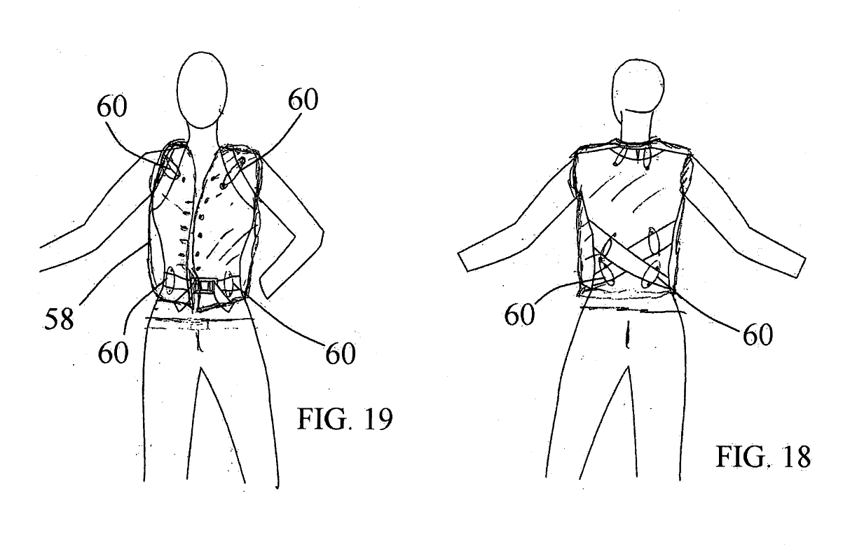

The buckle 28 is also capable of holding in place ends 14 and 16 of the elastic band 10, as seen in FIGS. 18 and 19, as the band is sandwiched between two or more layers of light weight fabric 58 containing built in loop guides 60 to keep the band 10 aligned in place without shifting within the material. In use, the garment will gently tug at the skin, muscles and upper torso to remind the body to be more erect and perpendicular to the force of gravity.

A method for improving a garment article with adjustable tensioning and releasable closure includes providing a socket and a plug each capable of receiving a first length of material and a second length of material respectively, with a first and second static straps each sewable onto the garment and a first and second length of material extending from within the garment for adjustable tensioning. Providing the socket and the plug each capable of being sewn onto the garment provides a releasable closure, and providing an outer garment that receives the article therein provides for adjustable tensioning and releasable closure thereof.

From the foregoing, it can be seen that there has been provided a unique buckle which provides for convenient and variable adjusting of the tensioning of an article from either end of the buckle while also providing a releasable closure. While a particular embodiment of the invention has been shown and described, it will be obvious to those skilled in the art that changes and modifications may be made without departing from the invention in its broader aspects. Therefore, the aim in the appended claims is to cover all such changes and modifications as fall within the true spirit and scope of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. An adjustable buckle for releasably joining opposite ends of an article, comprising:
a socket having an inlet and a resilient latch member projecting in the inlet;
a first non-fabric sewable static strap affixed to the socket opposite the inlet of the socket;
a plug having a resilient engaging member capable of releasably coupling with the latch member of the socket;
a second non-fabric sewable static strap affixed to an end of the plug opposite the resilient engaging member; and
said socket and said plug each capable of receiving a first length of material and a second length of material respectively, said first and second static straps each being sewable onto a garment with the first and second length of material extending from within the garment for adjustable tensioning.

2. An adjustable buckle as recited in claim 1, wherein said affixed comprises integrally molded thereto.

3. An adjustable buckle as recited in claim 1, wherein said socket and said plug are each capable of being sewn onto the garment to provide a releasable closure.

4. An adjustable buckle as recited in claim 3, wherein said first and second length of material extending from within the garment provide adjustable tensioning by either said first length of material or said second length of material.

5. An adjustable buckle as recited in claim 1, wherein said socket is a low profile hollow socket having a front shell with a resilient pressuring member formed in the front shell operable with said resilient latch member projecting in the inlet.

6. An adjustable buckle as recited in claim 5, wherein said plug is a low profile plug for engagement of said resilient engaging member with said resilient latch member of said socket projecting therein.

7. An adjustable buckle as recited in claim 5, wherein said socket comprises a back shell, a top shell, a bottom shell, and two side shells with said resilient latch member projecting therein.

8. An adjustable buckle as recited in claim 1, wherein said first and second static straps coupled to opposite ends of a band for adjustable tensioning of the band from either end with said socket and said plug each capable of being sewn onto the garment to provide a releasable closure.

9. An adjustable buckle as recited in claim 1, comprising an outer garment that receives the article therein for adjustable tensioning and releasable closure thereof.

10. A method for improving a garment article with adjustable tensioning and releasable closure comprising providing a socket and a plug each capable of receiving a first length of material and a second length of material respectively, the first and second static straps each being sewable onto the garment with the first and second length of material extending from within the garment for adjustable tensioning.

11. A method as recited in claim 10, wherein the socket and the plug are each capable of being sewn onto the garment to provide a releasable closure.

12. A method as recited in claim 10, comprising an outer garment that receives the article therein for adjustable tensioning and releasable closure thereof.

13. An adjustable buckle for releasably joining opposite ends of an article, comprising:
a socket having an inlet and a resilient latch member projecting in the inlet with a resilient pressuring button member formed thereon;
a first non-fabric sewable static strap affixed to the socket opposite the inlet of the socket;
a plug having a resilient engaging member capable of releasably coupling with the latch member of the socket;
a second non-fabric sewable static strap affixed to an end of the plug opposite the resilient engaging member; and
said socket and said plug each capable of receiving a first length of material and a second length of material respectively, said first and second static straps each being sewable onto a garment with the first and second length of material extending from within the garment for adjustable tensioning wherein said first and second length of material extend from within the garment to provide adjustable tensioning by either said first length of material or said second length of material.

14. An adjustable buckle as recited in claim 13, wherein said affixed comprises integrally molded thereto.

15. An adjustable buckle as recited in claim 13, wherein said first and second static straps coupled to opposite ends of a band for adjustable tensioning of the band from either end with said socket and said plug each capable of being sewn onto the garment to provide a releasable closure.

16. An adjustable buckle as recited in claim 13, comprising an outer garment that receives the article therein for adjustable tensioning and releasable closure thereof.
